(19) United States
(12) Patent Application Publication
(10) Pub. No.: US 2007/0043264 A1
(43) Pub. Date: Feb. 22, 2007

(54) Speculum

(75) Inventors: Ralph L. Gillis, Nashua, NH (US); Jo Ann Jorge, Ashland, MA (US); Joanne M. Krawczyk, Dunstable, MA (US); Marc A. Larochelle, Manchester, NH (US); Charlotte Richards, Newton, MA (US); Richard Ross, Newton, MA (US); Nicholas Want, Manchester, NH (US)

Correspondence Address: WOLF GREENFIELD & SACKS, PC, FEDERAL RESERVE PLAZA, 600 ATLANTIC AVENUE, BOSTON, MA, 02210-2206, US

(73) Assignee: Innovative Gynecological Solutions, LLC, Framingham, MA (US)

(21) Appl. No.: 11/418,816

(22) Filed: May 5, 2006

Related U.S. Application Data
(63) Continuation-in-part of application No. 11/137,290, filed on May 25, 2005.
(60) Provisional application No. 60/574,045, filed on May 25, 2004.

(51) Int. Cl. A61B 1/00

(52) U.S. Cl. 600/184

(57) ABSTRACT

A speculum for accessing a body cavity or other opening may include an insertion portion having a wall with a barrel-like shape that forms a passageway extending from a proximal end to a distal end of the insertion portion. The wall of the insertion portion may have one or more openings, e.g., a slot extending along the longitudinal axis of the passageway. An obturator may be received in the passageway, and an obturator support may position the obturator so as to contact the wall of the insertion portion at an opening. An illumination portion having one or more light pipes extending from a housing may be used with the speculum, e.g., to provide light in the passageway. The illumination portion may be received in a cavity of the speculum and provide illumination through one or more windows in a wall of the cavity.

This application is a continuation-in-part of U.S. application Ser. No. 11/137,290, filed May 25, 2005, and claims priority to U.S. Provisional Application 60/574,045, filed May 25, 2004. U.S. application Ser. No. 11/137,290 is hereby incorporated by reference in its entirety.

### BACKGROUND OF INVENTION

1. Field of the Invention

The present invention relates to specula, including a speculum for use in accessing an opening in a body, such as a vagina or a surgical wound.

2. Related Art

Specula are commonly used to access openings in a body, such as a vagina, ear canal, mouth, rectum, etc. One speculum configuration used with vaginas is a bivalve instrument having two blades that are movable relative to each other. The speculum is placed in the vagina with the blades collapsed together, and is advanced in different directions, e.g., toward the patient's abdomen and her back. Once the cervix is located, which in some individuals can be difficult due to anatomical variations of the cervix, body weight of the patient, laxity of the vaginal wall secondary to childbirth, height of the patient, and/or anxiety and increased muscle tone of the patient, one blade of the speculum is used to put traction on either the ventral aspect of the cervix (the part nearest the patient's abdomen), or on the dorsal aspect of the cervix (the part nearest the patient's back). This motion involves opening and closing the blades of the speculum, as well as pulling it back and forth, in and out of the vagina. The sensation of ‘digging’, moving, stretching, with an oftentimes cold speculum may be uncomfortable and/or embarrassing for the patient. Sometimes an examination performed with such a device is accompanied by tearing of the vaginal skin, pinching of the vulva (the outside part of the vagina), and/or bruising or bleeding of the cervix and vagina.

### SUMMARY OF INVENTION

In one aspect of the invention, a speculum is provided with no blades or other parts that are required to be moved to access a body opening. In one illustrative embodiment, a speculum includes an insertion portion having a barrel-like shape that may be inserted into the body opening with or without an obturator. Once positioned in the opening, the insertion portion may provide access to the opening via a passageway at the interior of the insertion portion. The passageway may be formed by an arcuate-shaped wall that extends from a proximal end to a distal end of the insertion portion. The wall may include a slot or other opening in a portion of the length of the insertion portion, or that extends along an entire length of the insertion portion. The insertion portion may also have a beveled end such that an end face of the wall at the distal end of the insertion portion is arranged at an angle to a longitudinal axis of the insertion portion, or otherwise be arranged so that one section of the distal end of the insertion portion extends distally further than other sections of the insertion portion.

The speculum may include an obturator adapted to be inserted in the passageway of the insertion portion and at least partially occlude the distal end of the passageway, e.g., to aid in insertion of the insertion portion into the body opening. An obturator support may position the obturator near the opening in the wall of the insertion portion, e.g., to help resist entry of tissue, fluids or other material into the passageway through the opening. For example, the obturator support may resiliently bias or otherwise force the obturator into contact with the wall of the insertion portion near the opening.

In one aspect of the invention, a speculum includes an insertion portion having a wall defining a passageway that extends along a longitudinal axis from a proximal end to a distal end of the insertion portion. The insertion portion wall may have an arcuate shape in a cross section transverse to the longitudinal axis, and at least one opening may be provided in the wall, such as a slot, hole, window or other. A handle portion may be located at the proximal end of the insertion portion, and be arranged to allow a user to manipulate the insertion portion during use. An obturator may also be provided which is constructed and arranged to fit within the passageway and at least partially occlude a distal end of the passageway, e.g., to ease insertion of the insertion portion into a cavity. At least one obturator support may be arranged in the passageway and position the obturator near the insertion portion at the at least one opening. Such positioning of the obturator near the insertion portion at the opening may help to occlude the opening, e.g., to help resist unwanted passage of fluid or tissue through the opening. At the same time, the obturator may not necessarily be in intimate contact with other portions of the wall of the insertion portion, e.g., to allow light, fluid, gases or other materials to pass in a space between the obturator and the wall.

In one embodiment, the opening includes a slot extending from the distal end to the proximal end of the insertion portion, and the obturator support biases the obturator into contact with the insertion portion at edges of the slot. The obturator support may include a support rib that extends along the longitudinal axis on the wall of the insertion portion opposite the slot. A scooped portion may be included at the distal end of the insertion portion that is curved toward the longitudinal axis and arranged to support the obturator when fully inserted into the passageway. Thus, the scooped portion may serve to bias the obturator toward an opening in the insertion portion as well.

Opposite lateral sides of the obturator may each include a concave groove that extends along the obturator longitudinal axis, e.g., to provide a pathway for light transmitted along the passageway. The handle portion may include an illumination pack cavity and illumination windows on opposite lateral sides of the proximal end of the passageway arranged so that light emitted by an illumination pack may be transmitted along the passageway.

In one embodiment, when the obturator is fully inserted into the insertion portion for use, an obturator longitudinal axis may be transverse to the insertion portion longitudinal axis, i.e., the obturator may be arranged at an angle with respect to the insertion portion.

In another aspect of the invention, an illumination device for use with a speculum is provided. The illumination device may include a housing arranged to at least partially enclose a power source and to fit within a cavity in a housing portion of a speculum. A light source may be connected to the power source and produce illumination, and a light pipe may be provided with a transmission portion that extends from the housing along a transmission axis along which illumination from the light source may travel. The transmission portion may have an angled end face with at least a portion arranged in a plane transverse to the transmission axis. In one embodiment, the housing may have a crescent shape with opposed ends, and a light pipe may be provided at each of the opposed ends. The transmission axis for the light pipes may be transverse to each other, e.g., arranged at an angle of about 60 degrees relative to each other. The angled end faces of the transmission portions may be arranged at an angle of about 50 degrees relative to each other.

In another aspect of the invention, a speculum may be provided with an illumination device. The speculum may include an insertion portion having a wall defining a passageway that extends along a longitudinal axis from a proximal end to a distal end of the insertion portion. The wall may have an arcuate shape in a cross section transverse to the longitudinal axis. A housing portion may be located at the proximal end of the insertion portion and include an illumination pack cavity and illumination windows on opposite sides, e.g., lateral sides, at the proximal end of the passageway. The housing portion may be located only at a lower region near the proximal end of the passageway so as to leave an upper region near the proximal end of the passageway free of the housing portion, e.g., to provide better access to the passageway. An illumination device may be arranged to fit, at least partially, in the illumination pack cavity and to emit illumination through the illumination windows and into the passageway.

These and other aspects of the invention will be apparent from the following description and claims.

### BRIEF DESCRIPTION OF THE DRAWINGS

Aspects of the invention are described below with reference to the following drawings in which like numerals reference like elements, and wherein:

FIG. 1 shows an exploded view of a speculum in accordance with aspects of the invention;

FIG. 2 shows an assembled view of the speculum of FIG. 1;

FIG. 3 shows a side view of the FIG. 1 speculum;

FIG. 4 shows a top view of the FIG. 1 speculum;

FIG. 5 shows a rear perspective view of the FIG. 1 speculum;

FIG. 6 shows a side view of the insertion portion of the FIG. 1 speculum;

FIG. 7 shows a top view of the insertion portion;

FIG. 8 shows a rear perspective view of the insertion portion;

FIG. 9 shows a close-up view of the distal end of the insertion portion of the FIG. 1 speculum;

FIG. 10 shows a sectional view along the line A-A of FIG. 5;

FIGS. 11 and 12 show front perspective and rear perspective views, respectively, of the illumination pack; and

FIG. 13 shows a sectional view along the line B-B of FIG. 5.

### DETAILED DESCRIPTION

Illustrative embodiments of a speculum for gynecological procedures including examinations, screenings, biopsies, and surgeries of the vagina, cervix and uterus are described below. However, aspects of the invention are not limited to the illustrative embodiments described, but rather may be incorporated, whether alone or in combination, with other aspects of the invention, in any suitable arrangement. In addition, aspects of the invention are not limited to use with respect to a vagina, but may be used with any body opening, such as a surgical wound, ear canal, mouth or throat, rectum, etc.

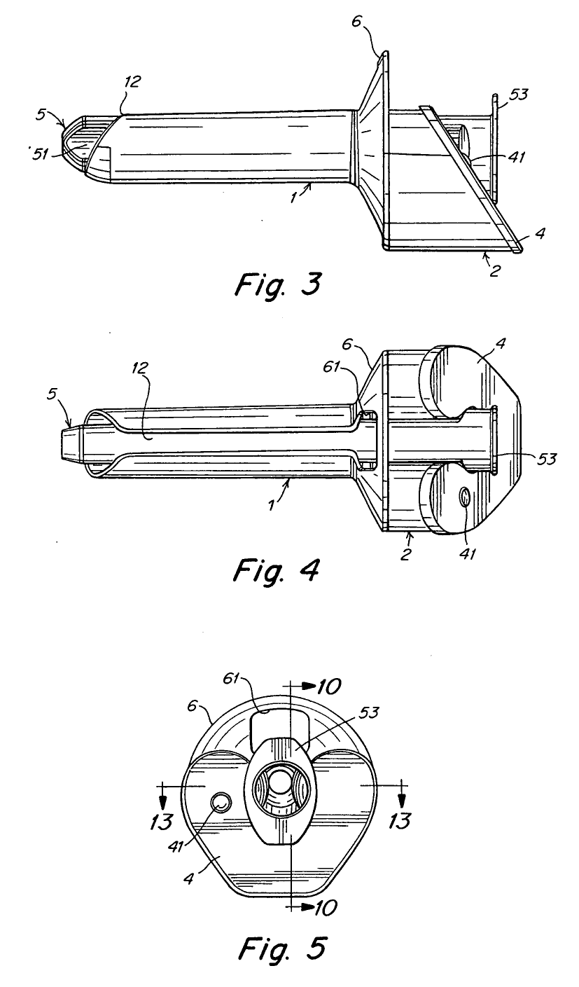

FIGS. 1-5 show exploded and assembled views of a speculum in accordance with aspects of the invention. In this embodiment, the speculum 100 includes an insertion portion 1 with a wall that defines an elongated passageway 11 that extends from a proximal end (near a flange portion 6 and a housing portion 2) to a distal end. Overall, the wall has an arcuate shape in a cross section transverse to a longitudinal axis of the passageway 11. As used herein, “arcuate” refers to shapes including a circular shape or portion of a circle, curves having portions with two or more radii (e.g., an oval or a portion of an oval), a shape formed by two or more straight portions (e.g., a polygon or a portion of a polygon), and other irregular shapes that approximate a curve. In this embodiment, the wall of the insertion portion 1 forms a barrel-shaped member that is approximately cylindrical, but the wall may form other suitable shapes, such as a conical or frustoconical barrel, a tube having an oval, irregular or polygonal cross-sectional shape, and so on. The inner and outer surfaces of the wall have similar cross sectional shapes in this embodiment, but may have different shapes, e.g., circular on the outer surface and oval on the inner surface, if desired. The outer and inner surfaces of the insertion portion 1 may be smooth, may have longitudinal ribs, or any other surface features as desired.

An opening may be provided in the wall of the insertion portion 1, e.g., to allow access to tissue at the body opening and/or to allow instruments to be provided at the distal end of the passageway 11. In this illustrative embodiment, the insertion portion I includes a slot 12 that extends along an entire length of the insertion portion 1, e.g., from a junction with the flange portion 6 to the distal end of the insertion portion 1. The slot or other opening 12 may be straight as shown, or may be arranged in any other suitable way, such as in a spiral, may have a varying width, size, shape, and so on. Moreover, the opening 12 need not extend to, or be located at, the distal and/or proximal ends of the insertion portion 1. Instead, the opening 12 may include a hole or open window through the wall at any suitable location between the proximal and distal ends of the insertion portion 1. (In some embodiments discussed below, the opening 12 need not be provided at all.)

The opening 12 may communicate with a notch 61 or other opening in the flange portion 6. The notch 61 and/and or opening 12 may provide space for surgical instruments, such as a tenaculum, to be passed through the passageway 11 and operated. In addition, the notch 61 may provide a more open area at an upper side of the flange portion 6 so as to allow easier access to the passageway 11 for viewing, instrument insertion, etc. The notch 61 may be provided in a section of the flange portion 6 that forms, in one aspect of the invention, a continuous band, i.e., forms a continuous pathway around the passageway 11 without breaks. The continuous band section may be joined to the proximal end of the insertion portion 1 and extend radially outwardly away from the passageway 11, e.g., to protect body portions around the opening from contact with surgical instruments and/or limit the insertion depth of the insertion portion 1.

The speculum 100 may include an obturator 5 or other device to aid in the insertion of the speculum 100 or provide other functions. In one aspect of the invention, the obturator 5 may be positioned relative to the opening 12 so as to partially occlude the opening 12. This may help prevent snagging, pinching or other contact of tissue at the opening 12 as the insertion portion 1 is manipulated in the body opening. For example, in this embodiment, the obturator 5 may be positioned in contact with the insertion portion 1 at the edges of the slot 12. This contact between the obturator 5 and the insertion portion 1 at the opening 12 may allow a practitioner to rotate, insert and/or remove the insertion portion 1 in the body opening while helping to prevent engagement of tissue with the insertion portion 1. Moreover, occlusion of the opening 12 may help reduce fluid flow through the opening 12. In one aspect of the invention, the obturator 5 may be positioned near or at the opening 12 in the insertion portion 1 by an obturator support that is located in the passageway 11 or elsewhere, as is discussed in more detail below. To aid in occlusion of the opening 12, the obturator 5 may have an external shape that, at least in part, closely conforms to or approximates the inner surface of the wall in the passageway 11, e.g., have a portion with a partial cylinder shape that extends into the passageway as shown.

In this illustrative embodiment, the speculum 100 includes an illumination portion 3 that provides light to illuminate an area of interest. The light may be transmitted through the passageway 11 and/or through one or more portions of the insertion portion 1 toward the distal end so as to illuminate a body portion that is located at the distal end of the insertion portion 1 and/or positioned within the passageway 11. In one aspect of the invention, opposite lateral sides of the obturator 5 each includes a concave groove 51 that extends along the obturator longitudinal axis. The concave grooves 51 may provide a space in the passageway 11 to allow light from the illumination portion 3 to pass toward the distal end of the passageway 11 when the obturator 5 is inserted in the insertion portion 1. It should be understood that although shown as having a smooth contour, the concave grooves 51 may have any suitable shape. The concave grooves 51 may also define a particular rotational position of the obturator 5 relative to the insertion portion 1. This may help ensure alignment of the concave grooves 51 with illumination pathways in the passageway 11.

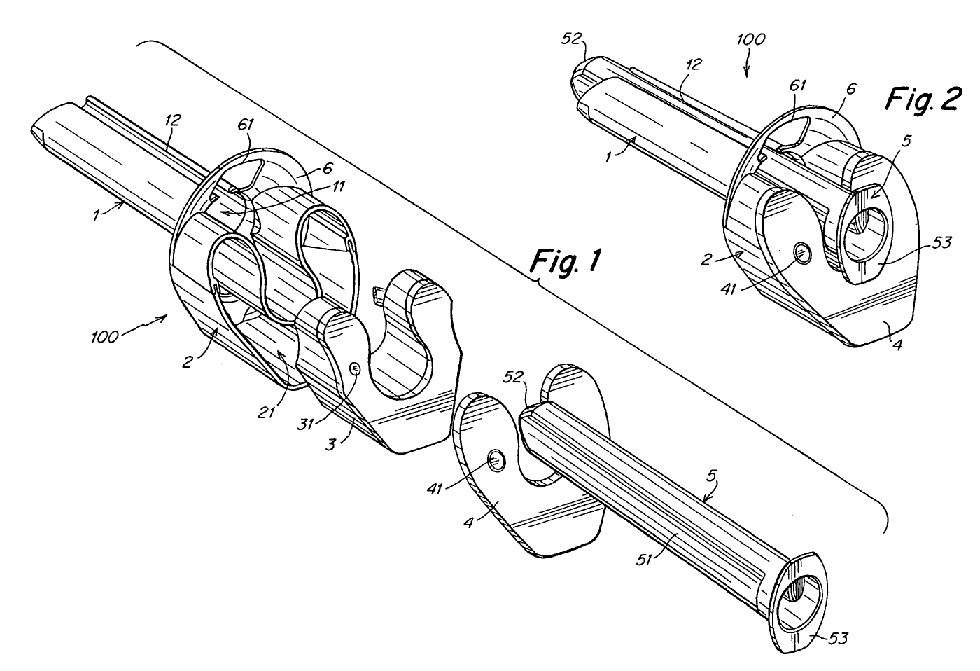

In one aspect of the invention, the illumination portion 3 may be arranged to fit within an illumination pack cavity 21 that is formed, at least in part, by a housing portion 2 that extends proximally from the flange portion 6. The cavity 21 may be arranged to provide a relatively open area above the housing portion 2, e.g., to allow better visual or physical access to the passageway 11. For example, the cavity may have a crescent-like shape as shown and be located only at a lower region (i.e., at lateral and/or bottom sides of the speculum) near the proximal end of the passageway so as to provide a relatively obstruction-free upper region at the proximal end of the passageway. The illumination portion 3 may be removably received in the cavity 21, and a cover 4 may seal over the open end of the cavity 21 as shown in FIG. 2 to retain the illumination portion 3 in place and help keep liquids and other materials from entering the cavity 21. The cover 4 may have an activation portion 41 to allow a user to actuate an on/off switch or other control feature(s) of the illumination portion 3 while the cover 4 is in place on the housing portion 2.

A handle for the speculum may be provided by an outer gripping surface of the housing portion 2 that is adapted to be gripped by a user to insert, rotate and otherwise manipulate the speculum 100. The gripping surface may extend proximally at a bottom side of the flange portion 6 and have an arcuate shape that is approximately concentric with the wall of the insertion portion 1. A top side of the flange portion 6 may be free of the housing portion 2 so as to permit access to the passageway 11. (Terms “top,” “bottom” and “side” are used only in a relative sense herein for ease of description. However, the use of these terms should not limit the way in which the speculum may be used, including various orientations of the speculum in a body opening.) For example, as can be seen in FIGS. 1-4, the housing portion 2 extends proximally relative to the passageway 11 and defines an arcuate gripping surface that is approximately concentric with the wall of the insertion portion 1. The gripping surface of the housing portion 2 may be relatively compact and may allow 360 degree rotation of the speculum 100 while in place in a body opening without interfering with the patient's legs or other body portions outside of the body opening, e.g., the housing portion or other gripping region of the speculum may extend no further radially than any other part of the flange portion 6. For example, in one illustrative embodiment, the housing portion 2 may define an arcuate gripping region having a diameter or maximum chord length of about 8 cm-9 cm or less. As will be understood, the housing portion 2 may include ribs, serrations or other features to enhance a user's grip and may be otherwise arranged to function well ergonomically.

Although in this embodiment, a handle is provided by the housing portion 2, in other embodiments, a handle may be provided by other features, such as a gripping region provided on the flange portion 6, by the illumination portion 3 (e.g., where no housing portion 2 is provided and the illumination portion 3 is connected to the flange portion 6), by a rod or other feature extending from the flange portion 6, and so on. In short, a handle may be provided for the speculum in any suitable manner.

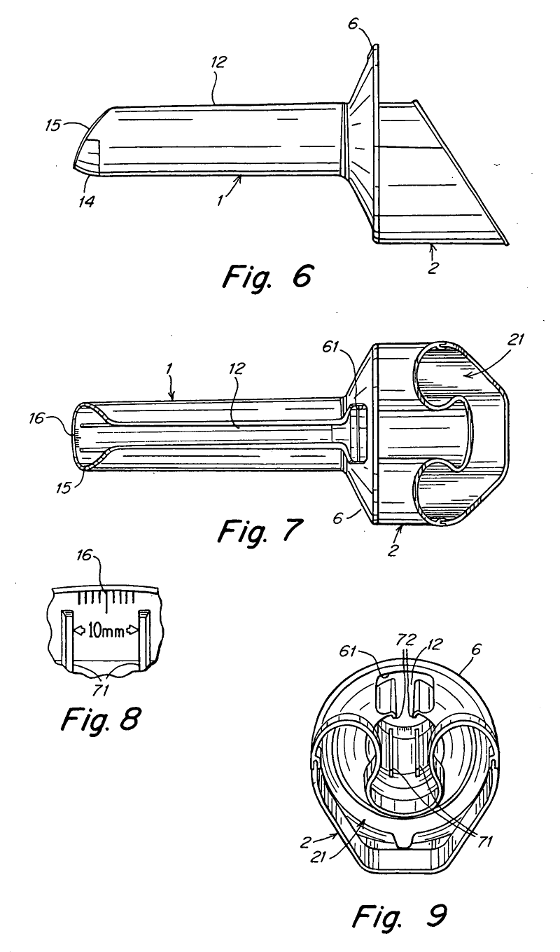

FIGS. 6-9 show views of the insertion portion 1 without the obturator 5 in place so that various aspects of the invention may be more clearly seen. As shown in FIG. 6, one part of the distal end of the insertion portion 1 may extend distally further than other portions of the distal end. For example, an end face 15 of the wall of the insertion member may beveled, i.e., be arranged at an angle to a longitudinal axis of the 25 passageway 11. This arrangement results in one portion of the end face 15 extending distally farther from the flange portion 6 than another portion of the end face 15. This arrangement may allow a user of the speculum 100 to manipulate portions of the body in or near the opening, such as a portion of a cervix, by contacting the body portion with the extended section of the insertion portion 1, or allow for better viewing or access. For 30 example, as shown in a side view of FIG. 6, the end face 15 of the insertion portion 1 may be arranged at an angle of approximately 45 degrees to a longitudinal axis of the passageway 11. In this embodiment, the bottom section of the insertion portion 1, i.e., the section opposite the slot 12, is somewhat longer than the upper section near the slot 12. Of course, it will be understood that the beveled end, if provided, may be arranged in other ways, e.g., so that a side section or the upper section of the insertion portion 1 (the portion near the slot 12) extends distally further than other sections of the insertion portion 1. In addition, it will be understood that the angle at which the end face 15 is arranged relative to the passageway 11 may be any suitable one, including 90 degrees, or perpendicular to the passageway. In this embodiment, the end face 15 has a slight convex curve as viewed in FIG. 6, but the end face 5 may have a concave curve or be flat. The edges of the end face 15 may be rounded, softened or otherwise configured to aid in insertion of the speculum 100 either with or without an obturator 5. Edge portions of the slot 12 and the end face 15 may be tapered, rounded or otherwise arranged so as to help avoid catching on tissue or other structures in the opening, such as when inserting the speculum 100 into the opening or rotating the speculum 100.

In one aspect of the invention shown in FIG. 6, the distal end of the insertion portion 1 may include a scoop 14, i.e., a portion at the distal end of the insertion portion 1 that is curved toward the passageway 11. The scoop 14 may aid in guiding the movement of the insertion portion 1 and/or serve as a dilator when inserting the speculum into a body opening. Also, as discussed below, the scoop 14 may support the obturator 5 when the obturator 5 is fully inserted into the passageway 11.

In another aspect of the invention, the speculum 100 may include a measurement feature that may be used to determine a size of one or more features in the body opening. For example, as shown in FIGS. 7 and 8, the scoop 14 or other part of the insertion portion 1 may include markings 16 by which the size of a feature may be gauged. In this embodiment, the scoop 14 may include lines or other features 16 at 1 millimeter increments along with a “10 mm” indicator that are molded into the insertion portion 1. Of course, it will be understood that any other suitable markings may be provided. For example, markings may be provided on the wall of the insertion portion 1 to indicate a depth that the insertion portion 1 is located in a body opening. Near, or in addition to, areas including markings, may be included chemical or other indicators to provide an indication of a particular chemical or other substance of interest.

As discussed above, in accordance with one aspect of the invention, the speculum may include an obturator support that serves to position the obturator near an opening in the insertion portion. By positioning the obturator near the opening (e.g., in contact with or close approximation to the wall of the insertion portion at the opening), the obturator may at least partially occlude the opening, helping to resist tissue, fluid or other material from entering the opening. This may help prevent pinching, snagging or other engagement with tissue, such as when the speculum is inserted into a body cavity and/or rotated within the cavity. In this illustrative embodiment, the obturator support includes two pairs of ribs that extend along the longitudinal axis of the passageway 11. In this embodiment, a pair of support ribs 71 extend along a portion of the inner wall of the insertion portion 1 that is opposite the slot 12. The support ribs 71 are arranged to contact a lower side of the obturator 5 and urge the obturator 5 to move upwardly toward the slot 12. The space between the support ribs 71 (measured in a direction perpendicular to the ribs 71) may be arranged so that the lower cylindrical portion of the obturator 5 is cradled between the ribs 71. In addition, or alternately, the lower side of the obturator 5 may include a groove or other feature arranged to engage with one or both of the ribs 71, e.g., to help prevent rotation of the obturator 5 about its longitudinal axis.

The obturator support may also include a pair of slot ribs 72 arranged at edges of the slot 12 (or other opening). The slot ribs 72 may extend inwardly from the wall of the insertion portion at the edges of the slot 12 and be arranged to contact the upper surface of the obturator 5. Like the support ribs 71, the slot ribs may have any length, size, width or other arrangement. Also, the obturator 5 may include a groove or other feature to engage with the slot ribs 72, e.g., to help maintain contact between the ribs 72 and the obturator 5 and resist tissue capture. The support ribs 71, the slot ribs 72 and/or portions of the obturator 5 may be resilient to allow resilient contact between the obturator 5 and the insertion portion 1 at or near the opening 12.

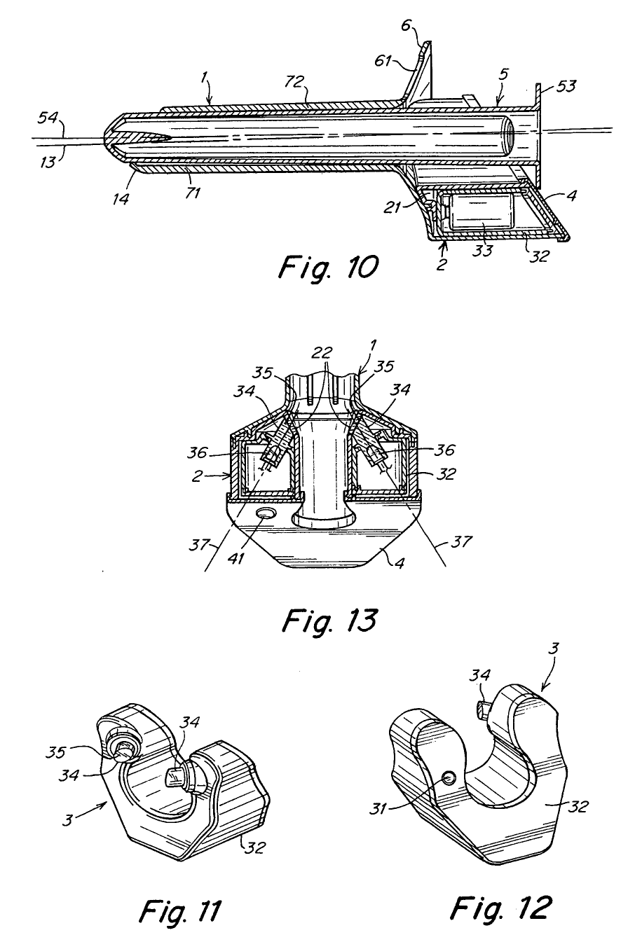

FIG. 10 shows a cross sectional view along the line A-A in FIG. 5 that passes through one of the slot ribs 72 and one of the support ribs 71, which in this embodiment are each molded integrally with the insertion portion 1. Of course, it will be understood that the ribs 71 and 72 may be formed in other ways, such as by separate elements that are attached to the insertion portion 1. In this embodiment, a height of the ribs 71 and 72 (i.e., a distance that the ribs extend inwardly from the inner wall surface of the insertion portion 1) tapers in a direction along the longitudinal axis of the passageway 11. For the support ribs 71, the height of the ribs 71 tapers (i.e., becomes smaller) in a direction from the distal end to the proximal end of the passageway 11. For the slot ribs 72, the height of the ribs 72 tapers in a direction from the proximal end to the distal end of the passageway 11. As a result, and in accordance with an aspect of the invention, a longitudinal axis 54 of the obturator 5 is arranged to be transverse to a longitudinal axis 13 of the passageway 11. In this illustrative embodiment, this arrangement places the obturator 5 at an angle relative to the insertion portion 1, i.e., generally sloping upwardly relative to the insertion portion 1. The scoop 14 may support the obturator 5 in this way by contacting the lower surface of the obturator 5 near its distal end 52. The support ribs 71 and/or the scoop 14 may urge the obturator 5 to move upwardly and into contact with the insertion portion 1 near the slot 12 (in this case into contact with the slot ribs 72).

It should be understood that the obturator support feature that positions the obturator near an opening in the wall of the insertion portion is not limited to the rib and/or scoop arrangement described above. For example, the support ribs 71 may be replaced with the scoop 14 only and/or one or more tabs, bumps, or other features that serve to lift the obturator 5 upwardly from the bottom surface of the inner wall of the insertion portion 1. Alternately, the obturator support may include a rail or other feature on the sides of the inner wall of the insertion portion 1 that engage with the grooves 51 of the obturator 5 and position the obturator 5 with respect to the opening 12 as desired. In another embodiment, the obturator support may include one or more spring fingers, a leaf spring, spring pin or other feature on the obturator 5 and/or in the passageway 11 that serves to urge the obturator into a desired position with respect to the opening in the wall of the insertion portion 1. In short, the obturator support may include any suitable arrangement to urge the obturator 5 into contact with or close proximity to an opening 12 in the insertion portion 1.

Other aspects of the invention relate to specific features of an illumination pack 3 for the speculum 100. In one embodiment shown in FIGS. 11 and 12, the illumination pack 3 includes a housing 32 that has a generally crescent shape, e.g., to fit in the illumination pack cavity 21. (In some embodiments, the housing portion 2 and/or cavity 21 may not be provided. In such a case, the illumination pack 3 may mate with the flange portion 6 or another portion of the speculum to provide illumination, as desired.) It should be understood that the housing 32 may have any suitable shape, and need not necessarily have a crescent shape to fit in the cavity 21, e.g., the housing 32 may include two or more separable parts that fit together in the cavity 21. The housing 32, which may be made of plastic, metal, a composite or any suitable combination of materials, may contain a power source, such as a battery 33 as seen in FIG. 10. The housing 32 may also include other components, such as electronic circuitry, a microprocessor, indicator lights or other display elements, an on/off switch 31 or other control element, etc. The housing 32 may be sealed, e.g., include sealed plastic walls to prevent liquids or other foreign materials from entering the housing, and/or may be made so that the housing may be opened to allow repair, battery replacement, etc. and closed to reseal the housing. The illumination portion 3 may include a device that enables the illumination portion 3 to be turned on when attached to the speculum 100, but otherwise disables the illumination portion 3. For example, the illumination portion 3 may include a switch that is closed when the illumination portion is connected to the speculum 100, but is open when the illumination portion is separated from the speculum. This may help avoid inadvertently leaving the illumination portion on when not in use. The illumination portion 3 may also include a “dimming” feature or other arrangement to control the intensity or other features of the light emitted. The illumination portion 3 may also include a red indicator light or other display that indicates a low battery condition, illumination malfunction or other information. In one embodiment, the illumination portion 3 may be connected to an exterior power source, such as a power cord plugged into a wall outlet or other. Such an arrangement may charge a battery in the illumination portion 3 and/or provide power to the illumination source(s).

Light provided by the illumination portion 3 may be transmitted by light pipes 34 or other structures that are attached to, or otherwise incorporated with the housing 32. In this illustrative embodiment, a pair of light pipes 34 are mounted at opposed ends of the housing 32 and extend away from the housing 32 and generally toward each other. The light pipes 34 include approximately cylindrical bodies that are attached at one end to the housing 32 and at an opposite end have an angled end face 35. As can be seen in FIG. 13, which is a cross sectional view along the line B-B in FIG. 5, the light pipes 34 are arranged so that the angled end faces 35 abut or are near to illumination windows 22 formed in the walls of the illumination pack cavity 21 when the illumination portion 3 is in the cavity 21 and the cover 4 is in place. The illumination windows 22 may be formed integrally with the housing portion 2, and include a transparent material through which light from the light pipes 34 may pass, but otherwise allows for sealing of the cavity 21. (In other embodiments, the windows 22 may be formed by openings in the housing portion 2 or other part of the speculum.) The close proximity of the end faces 35 to the windows may help reduce light loss through absorption, scattering or other. In this embodiment, the light pipes 34 each have a light source, e.g., an LED, embedded in the light pipe cylindrical body so that light emitted generally travels along a transmission axis 37 and through the illumination windows 22. In this embodiment, the transmission axes 37 are arranged at an angle of about 60 degrees relative to each other, and the angled end faces 35 lie in planes that are at an angle of about 50 degrees relative to each other. (In other embodiments, the angle between the transmission axes 37 may fall in a range of about 50 degrees to about 70 degrees, and the angle between the planes of the angled end faces 35 may fall in a range of about 40 degrees to about 60 degrees.) Light emitted from the illumination windows 22 may pass into the passageway 11 and travel distally down the passageway 11, e.g., in a space bounded by the insertion portion 1 and the concave grooves 51 of the obturator 5. The light pipes 34 and illumination windows 22 may be arranged so that light is bent, dispersed, filtered, focused or otherwise operated on so that desired illumination characteristics are provided, e.g., to illuminate a subject at the distal end of the passageway 11. The insertion portion 1 may be made of a transparent or translucent material that transmits light from the illumination portion 3 to an area of interest. In addition to enabling the transmission of light, transparent sections of the insertion portion 1 may allow a user to view features of the body opening through the wall of the insertion portion 1. Clear lubricant or other liquid material may aid in viewing such features. Alternately, the insertion portion 1 may be made of an opaque material and arranged to reflect light. Lenses, diffusers, light pipes, coatings or other optical features incorporated with the insertion portion 1 may serve to direct light and uniformly illuminate the area of interest.

Portions of the speculum 100, such as the insertion portion 1 and/or the flange portion 6, may be made to be disposable, for example being molded as a single unitary part of polycarbonate, crystal polystyrene or any other suitable material, or combination of materials, whether opaque, transparent or translucent. Portions of the speculum may be molded together in a unitary form, or may be made separately and joined together, e.g., by an adhesive, mechanical fasteners, welding, removable attachment, and so on. For example, the housing portion 2, the insertion portion 1/flange portion 6 may be made separately and then joined together. Various portions of the speculum may be made of any suitable material, such as plastics, composites, metals, and so on.

Portions of the speculum may be coated or treated in different ways, such as applying a charcoal or other coating to the interior wall of the insertion portion to help make the speculum more resistant to damage by laser or other light forms. Coatings or other treatments may be used to make the speculum more resistant to damage by heat, chemicals, abrasion, etc.

The obturator 5 may have a blunt, tapered, cone-shaped or otherwise suitably formed distal end 52. The distal end 52 may have an opening (not shown) that is suitable for performing certain functions, such as to visualize and/or perform procedures on the cervical os, fetal scalp, or other. For example, the distal end 52 of the obturator 5 around the opening may be positioned against the fetal scalp, and the scalp may be accessed for certain examinations. The opening in the distal end 52 of the obturator 5 may also be used to guide instruments (such as a brush or other sampling device) into the cervix or uterus, e.g., for endometrial biopsy, examination of the fetal head (for obtaining scalp pH), fetal cell collection, meconium, chorionic villus sampling, embryo transfer, endocervical curettage, and hysteroscopy.

The obturator 5 may also be used to stop uterine bleeding from the cervix, and may be made of a clear or transparent material (e.g., plastic). The obturator 5 (or the insertion portion 1) may also be formed of suitable color, transparent materials to enhance visualization and diagnosis of certain conditions, such as cervicitis, dysplasia, inflammation, developmental anomalies, hypervascularity, lacerations, bruises and discharges, etc. As discussed above, the obturator S may transmit illumination, whether for therapeutic or visualization purposes. The obturator 5 may include a stop, ring or other feature (e.g., a flange 53) to limit the insertion depth of obturator into the passageway 11, or may be arranged so as to pass entirely through the passageway 11. Without a flange 53 or similar, the obturator 5 may be first inserted into the body opening alone without the insertion portion 1. Thereafter, the insertion portion 1 may be slid over the proximal end of the obturator 5 and inserted into the opening while the obturator 5 remains in place.

In one embodiment when using a speculum in accordance with the invention, the insertion portion 1 and obturator 5 may be arranged so the obturator 5 is inserted into the passageway 11 as shown in FIG. 2 and the speculum advanced into the vagina. Clear water soluble or other suitable lubricant may be placed along the outside of the speculum, e.g., to enhance visualization. Once the cervix is located (e.g., by visualizing the cervical os through a distal opening in the obturator 5), the obturator 5 may be pulled back from within the insertion portion 1 and be partly, or completely withdrawn. The insertion portion 1 may be rotated until the distal end of the cervix is located and pulled into view. Rotation of the insertion portion 1 may take place with or without the obturator in place. Since the beveled end 15 of the speculum 100 may increase the surface area of the end of the insertion portion 1, procedures such as a pap smear may be accomplished. A smaller speculum may thus be employed using this configuration because the sides of the speculum hold the lateral walls of the vagina from obstructing the view of the person during procedures such as a pap smear.

In another aspect of the invention, the continuous sidewall configuration of the insertion portion may be an improvement over traditional specula having a bivalve configuration. The nearly continuous sidewall (with properly shaped and sized openings for, e.g., taking biopsies) may prevent vaginal or other tissue from moving into the line of sight and obscuring the examiner's view, thus improving the view down the longitudinal axis of the speculum. This contrasts with a traditional bivalve speculum, which has no continuous sidewalls to keep tissue from hindering the view down the longitudinal axis of the device. There is also the possibility that a vaginal examination may be performed in the lateral position instead of the dorsal lithotomy position because the side walls of the vagina are not obstructing the view.

As discussed above, different lighting arrangements may be used to help treat or visualize different conditions at the body opening. For example, illumination carried by the obturator 5 and/or insertion portion 1 (whether visual and/or non-visible light) may neutralize or prevent chemical changes that occur or may occur in the body opening, may neutralize harmful chemicals (such as lowering cytokines or inflammatory chemicals in a vagina), activate or enhance the action of photosensitive or photo-activated chemicals (such as peroxide chemicals), activate chemicals that form a barrier for viral, chemical or bacterial invasion, help identify pathological conditions or stages of labor (with or without the use of chemical indicators), such as rupture of fetal membranes, premature labor or gestational age, etc. The obturator 5 may be used as an applicator for medication, e.g., by introducing the medication through an opening at the distal end of the obturator, or by apply the medication to the distal end of the obturator while removed from the insertion portion, and then inserting the treated obturator into the insertion portion. For example, therapeutic use of light and/or activated creams/chemicals may be used to treat early onset of labor that may be caused by tumor necrosis factor which causes changes at the cervix.

The speculum may be available in various sizes (e.g., longer or shorter than conventional bivalve specula) to accommodate differences between patient anatomies, with a larger size available to also address procedural difficulties encountered in the operating room as well as the office setting. For example, the length of the insertion portion 1 may range from approximately 7-20 cm, while the length of the obturator 5 may range from approximately 8-20 cm. The outside diameter of the insertion portion 1 may range from approximately 1.5-5 cm.

Furthermore, the speculum may include a magnification component such as one or more lenses that allows for one or more magnification settings that may be used to facilitate examinations, e.g., enlarge the view of an area of interest in the passageway 11 for examination purposes.

In another aspect of the invention, the insertion portion 1 wall may have one or more openings in addition to, or in place of, the slot 12. These openings may be located anywhere along the length of the insertion portion 1 and inward from the proximal or distal end. The openings may have any suitable shape, such as oval, round or otherwise shaped, in various positions, making rotation of the instrument and procedures such as biopsy of the vaginal walls and cervix possible. In the embodiment where the rest of the insertion portion 1 is constructed of clear plastic, the use of an opening of a relatively small size may eliminate or limit the resultant blood loss from the area of a biopsy or other procedure, thus preventing obstruction of the view.

In one embodiment, even though the speculum in accordance with aspects of the invention may end up being slightly longer than a typical bivalve speculum, because light may be directed near the distal end and along the walls of the vagina, the instrument may not have to be inserted all the way up to the flange portion 6. On the other hand, a conventional speculum has to be inserted all the way to the handle end to minimize the need to open the blades any more than necessary. In a very short patient, there is a sensation of having very deep placement of the bivalve speculum to achieve this goal. An embodiment of the invention may be more versatile than a traditional bivalve speculum, which may require the doctor to use a much smaller speculum, such as a pediatric size, thereby potentially limiting the discomfort of a shorter patient. At the same time, the use of a smaller bivalve speculum may result in losing quality in terms of the ability to visualize and conduct procedures in the vagina, cervix and uterus. An aspect of the invention provides a more versatile speculum that may be used with patients of varied sizes, without losing any ability to conduct a thorough examination.

In another embodiment, the speculum 100 may be fitted with a minicam or other image capturing device for visualization and/or recording the appearance of the body opening being examined. The obturator may be fitted with an image capturing device near its distal end to visualize and record the appearance of the walls of the opening. Also, an image capturing device may be placed on a wand, or other device, and the walls of the body opening could be inspected in a 360 degree manner as the insertion portion of the speculum is withdrawn.

The speculum may also be arranged to provide different function. For example, the insertion portion 1 may include one or more channels that are used to evacuate smoke or fluids from the opening, or to provide a source of irrigation to the opening. The channels may be formed integrally with the insertion portion 1 or attached to the insertion portion 1. The channels may be arranged at any location on the speculum, such as at a bottom, side and/or top portion. One or more connection points may be provided to facilitate attachment of tubes or other sources of vacuum or irrigation fluid.

Specula arranged in accordance with aspects of the invention may be used in a variety of different ways, with a variety of different tools and a variety of different body openings. For example, the speculum may be used in procedures involving the use of a sharp toothed tenaculum, a long Allis clamp, crushing clamps, a scalpel or suturing instrument, forceps of different types, a curette, one or more dilators, hot loop endocervical wires, pap smear tools, a colposcope lens/light or other related device, one or more sponges, chemicals or other agents for treating different medical conditions, a pipette for sampling chorionic villous tissue, a fetal scalp electrode or fetal oxygenation detector, one or more catheters including balloon-tipped catheters and hysterosalpingogram catheters, intrauterine device insertion tools, vaginal pessaries, contraceptive diaphragms or other devices, intrauterine insemination catheters or embryo transfer catheters, ultrasound probes, a syringe, a biopsy needle, endometrial ablation devices, and others. Body openings that may be used with the specula include the vagina, cervix (e.g., for colposcopy), rectum, mouth, throat, ear, a surgical opening, and so on. Such openings may be present in any animal, including human or other mammal, reptiles, avian, or other.

While the invention has been described with reference to various illustrative embodiments, the invention is not limited to the embodiments described. It is evident that many alternatives, modifications and variations of the embodiments described will be apparent to those of ordinary skill in the art. Accordingly, embodiments of the invention as set forth herein are intended to be illustrative, and not limiting the scope of the invention. Various changes may be made without departing from the scope of the invention.

What is claimed is:

1. A speculum comprising:
an insertion portion having a wall defining a passageway that extends along an insertion portion longitudinal axis from a proximal end to a distal end of the insertion portion, the insertion portion wall having an arcuate shape in a cross section transverse to the insertion portion longitudinal axis, the insertion portion including at least one opening in the wall;
a handle at the proximal end of the insertion portion, the handle arranged to allow a user to manipulate the insertion portion during use;
an obturator constructed and arranged to fit within the passageway and at least partially occlude a distal end of the insertion portion passageway to ease insertion of the insertion portion portion into a cavity, the obturator extending along an obturator longitudinal axis; and
at least one obturator support in the passageway that positions the obturator near the insertion portion at the at least one opening.

2. The speculum of claim 1 wherein the at least one opening includes a slot extending from the distal end to the proximal end of the insertion portion, and the obturator support biases the obturator into contact with the insertion portion at edges of the slot.

3. The speculum of claim 2, wherein the at least one obturator support includes a support rib extending along the insertion portion longitudinal axis on a portion of the insertion portion wall opposite the slot.

4. The speculum of claim 3, further comprising a pair of slot ribs extending from the insertion portion wall on each side of the slot and that have a height that tapers from the proximal end to the distal end of the passageway, each of the slot ribs contacting the obturator when the obturator is fully inserted into the passageway.

5. The speculum of claim 3, wherein a height of the support rib tapers from the distal end to the proximal end.

6. The speculum of claim 1, wherein the obturator includes a blunt end at a distal end of the obturator, and wherein the blunt end extends from the distal end of the insertion portion when the obturator is fully inserted into the passageway.

7. The speculum of claim 1, wherein the at least one obturator support comprises a scooped portion at the distal end of the insertion portion that is curved toward the insertion portion longitudinal axis and is arranged to support the obturator when fully inserted into the passageway.

8. The speculum of claim 1, wherein opposite lateral sides of the obturator each includes a concave groove that extends along the obturator longitudinal axis.

9. The speculum of claim 1, further comprising a housing portion at a proximal end of the insertion portion that includes a crescent-shaped illumination pack cavity and illumination windows on opposite lateral sides of the proximal end of the passageway.

10. The speculum of claim 9, further comprising an illumination device arranged to fit within the illumination pack cavity and to emit illumination through the illumination windows and into the passageway.

11. The speculum of claim 9, further comprising a cover arranged to mate with the housing portion and cover the illumination pack cavity.

12. The speculum of claim 1, wherein the at least one obturator support extends inwardly from the insertion portion wall and is arranged to space the obturator away from at least a portion of the wall of the insertion portion.

13. The speculum of claim 1, wherein the obturator includes an opening at a distal end of the obturator.

14. A speculum comprising:
an insertion portion having a passageway extending along an insertion portion longitudinal axis from a proximal end to a distal end of the insertion portion, the insertion portion having an arcuate shape in a cross section transverse to the insertion portion longitudinal axis;
a handle at the proximal end of the insertion portion, the handle arranged to allow a user to manipulate the insertion portion during use;
an obturator constructed and arranged to fit within the passageway and at least partially occlude a distal end of the insertion portion passageway to ease insertion of the insertion portion into a cavity, the obturator extending along an obturator longitudinal axis; and
wherein when the obturator is fully inserted into the insertion portion for use, the obturator longitudinal axis is transverse to the insertion portion longitudinal axis.

15. The speculum of claim 14, wherein the insertion portion includes at least one rib extending inwardly from an inner surface of the insertion portion, the at least one rib arranged to support the obturator in the passageway.

16. The speculum of claim 15, wherein the at least one rib extends in a direction generally along the insertion portion longitudinal axis.

17. The speculum of claim 16, comprising a pair of ribs extending along opposite inner surfaces of the insertion portion.

18. The speculum of claim 17, wherein a height that each of the ribs extends from the inner surface of the insertion portion varies from the proximal end to the distal end.

19. The speculum of claim 18, wherein the height of one of the ribs is larger near the proximal end, and the height of the other of the ribs is larger near the distal end.

20. The speculum of claim 19, wherein the insertion portion includes a slot that extends from the distal end toward the proximal end, and one of the ribs is located adjacent the slot.

21. The speculum of claim 14, wherein a scooped portion of the insertion portion at the distal end of the insertion portion is curved toward the insertion portion longitudinal axis and is arranged to support the obturator when fully inserted into the passageway.

22. An illumination device for use with a speculum, comprising:
a housing arranged to at least partially enclose a power source and to fit within a cavity in a housing portion of a speculum;
a light source connected to the power source and adapted to produce illumination; and
a light pipe including a transmission portion extending from the housing along a transmission axis along which illumination from the light source travels, the transmission portion having an angled end face with at least a portion arranged in a plane transverse to the transmission axis.

23. The illumination device of claim 22, wherein the housing has opposed ends, and the illumination device includes a light pipe at each of the opposed ends, each light pipe including a transmission portion extending from the housing along a transmission axis along which illumination from the light source travels, the transmission portion having an angled end face with at least a portion arranged in a plane transverse to the transmission axis, and wherein the transmission axes of the light pipes are transverse to each other.

24. The illumination device of claim 23, wherein the transmission axes are arranged at an angle of about 60 degrees relative to each other.

25. The illumination device of claim 23, wherein the planes for the angled end faces are arranged at an angle of about 50 degrees relative to each other.

26. The illumination device of claim 22, wherein the transmission portion includes a transparent cylindrical body, and the angled end face is formed by an angled end face of the transparent cylindrical body.

27. The illumination device of claim 26, wherein an end of the transparent cylindrical body of each light pipe opposite the angled end face is fixed to the housing.

28. The illumination device of claim 26, wherein each transparent cylindrical body includes a light source embedded therein.

29. A speculum comprising:
an insertion portion having a wall defining a passageway that extends along an insertion portion longitudinal axis from a proximal end to a distal end of the insertion portion, the insertion portion wall having an arcuate shape in a cross section transverse to the insertion portion longitudinal axis;
a housing portion at the proximal end of the insertion portion including an illumination pack cavity and illumination windows on opposite sides of the passageway at the proximal end, the housing portion being located only at a lower region near the proximal end of the passageway so as to leave an upper region near the proximal end of the passageway free of the housing portion; and
an illumination device arranged to fit at least partially in the illumination pack cavity and to emit illumination through the illumination windows and into the passageway.

30. The speculum of claim 29, wherein the illumination device includes a housing with opposed ends, a light source, and a light pipe at each of the opposed ends of the housing, each light pipe including a transmission portion extending from the housing along a transmission axis along which illumination from the light source travels, the transmission portion having an angled end face with at least a portion arranged in a plane transverse to the transmission axis.

31. The illumination device of claim 30, wherein the transmission axes are arranged at an angle of about 60 degrees relative to each other.

32. The illumination device of claim 30, wherein the planes for the angled end faces are arranged at an angle of about 50 degrees relative to each other.

33. The illumination device of claim 30, wherein the transmission portion includes a transparent cylindrical body, and the angled end face is formed by an angled end face of the transparent cylindrical body.

34. The illumination device of claim 33, wherein an end of the transparent cylindrical body of each light pipe opposite the angled end face is fixed to the housing.

35. The illumination device of claim 33, wherein each transparent cylindrical body includes a light source embedded therein.

36. The illumination device of claim 30, wherein the transmission axes are arranged at an angle of between about 50 to 70 degrees relative to each other, and the planes for the angled end faces are arranged at an angle of between about 40 to 60 degrees relative to each other.

37. The illumination device of claim 29, further comprising an obturator having an obturator longitudinal axis and constructed and arranged to fit within the passageway and at least partially occlude a distal end of the insertion portion passageway to ease insertion of the insertion portion into a cavity, wherein opposite lateral sides of the obturator each includes a concave groove that extends along the obturator longitudinal axis and is adapted to allow illumination to pass from the illumination windows through the passageway with the obturator arranged in the passageway.

38. The speculum of claim 29, further comprising a cover arranged to mate with the housing portion and cover the illumination pack cavity.
